(19) United States
(12) Patent Application Publication
(10) Pub. No.: US 2010/0312108 A1
(43) Pub. Date: Dec. 9, 2010

(54) Tee-assisted cardiac resynchronization therapy with mechanical activation mapping

(76) Inventors: Harold M. Hastings, Garden City, NJ (US); Scott L. Roth, East Hills, NY (US)

Correspondence Address: PROSKAUER ROSE LLP, One International Place, Boston, MA, 02110, US

(21) Appl. No.: 12/800,833

(22) Filed: May 24, 2010

Related U.S. Application Data
(60) Provisional application No. 61/180,653, filed on May 22, 2009.

(51) Int. Cl. A61B 8/14; A61N 1/362

(52) U.S. Cl. 600/439

(57) ABSTRACT

A method for generating an enhanced ultrasound display including the steps of capturing a set of ultrasound image frames and identifying pixels in the captured set of frames that correspond to a structure. The method also includes generating a set of output frames. Each output frame within the set of output frames is generated by (a) selecting a first frame of the captured set; (b) selecting a second frame of the captured set, wherein the second frame is subsequent in time to the first frame; (c) coloring pixels of the first frame that correspond to the structure a first color; (d) coloring pixels of the second frame that correspond to the structure a second color; and (e) overlaying the colorized first frame and the colorized second frame to generate the output frame. The method also includes displaying the output frames.

### RELATED APPLICATIONS

This application claims the benefit of U.S. Provisional Application Ser. No. 61/180,653 entitled “Tee-Assisted Cardiac Resynchronization Therapy with Mechanical Activation Mapping,” filed on May 22, 2009. The entire disclosure of U.S. Provisional Application Ser. No. 61/180,653 is incorporated herein by reference.

### TECHNICAL FIELD

The invention relates to cardiac synchronization therapy and to highlighting motion on imaging displays including but not limited to ultrasound displays.

### BACKGROUND

In a normal sinus beat, the left ventricle (“LV”) contracts in a coordinated manner, efficiently ejecting a significant fraction of the blood in its cavity. This behavior arises from: coordinated (i.e., appropriately synchronized) electrical stimuli from the His-Purkinje network; good electrical conduction in the ventricle, spreading electrical activation in an appropriate, coordinated way throughout the ventricle (endpoint 1); and finally good electrical-mechanical coupling, translating coordinated electrical activation into appropriate, coordinated mechanical activation (endpoint 2), resulting in efficient ejection (endpoint 3).

On the other hand, mechanically dyssynchronous contraction of the LV results in inefficient pumping. Mechanical dyssynchrony may arise from uncoordinated electrical stimulation, areas of low conduction, or lack of good electrical-mechanical coupling. Cardiac resynchronization therapy (“CRT”) aims to correct dyssynchrony by applying suitably timed electrical stimuli to one or both ventricles.

In conventional CRT, an electrode is guided into a position inside or outside the left heart, typically using an anatomical imaging method such as fluoroscopy or thoracoscopy. Electrical pulses are then applied to the electrode to improve the synchronization of the heart muscle (and thereby improve the heart's pumping performance). Unfortunately, the placement of electrodes that are positioned using current methods is sub-optimum in many cases, as is the improvement in synchronization.

### SUMMARY

One aspect of the invention relates to a method for positioning an electrode for improved cardiac synchronization. The method includes inserting an ultrasound probe into a patient's esophagus. The ultrasound probe is used to obtain a first set of images of the patient's heart. The method further includes determining, based on the first set of images, a first portion of the heart whose motion is delayed with respect to other portions of the heart. A first electrode is positioned at a first location near the first portion of the patient's heart. Pulses are applied to the first electrode that are timed with respect to beating of the heart to attempt to advance, in time, the motion of the portion of the heart. The method also includes using the ultrasound probe to obtain a second set of images of the patient's heart. The method further includes determining, based on the second set of images, whether motion of the first portion of the heart is sufficiently synchronized with respect to other portions of the heart. If it is determined, based on the second set of images, that the motion of the first portion of the heart is not sufficiently synchronized with respect to other portions of the heart, the first electrode is re-positioned at a second location and pulses are applied to the first electrode that are timed with respect to beating of the heart to attempt to advance, in time, the motion of the portion of the heart.

In some embodiments, the method further includes the step of processing the first set of images and the second set of images to highlight portions of the heart that have moved between two successive images in the first set of images. In some embodiments, the first set of images and the second set of images are enhanced with at least two colors. In some embodiments, the processing includes detecting a difference between two successive images in the first set of images.

In some embodiments, determining, based on the first set of images, a first portion of the heart whose motion is delayed with respect to other portions of the heart includes the step of distinguishing between a motion generated by a local area contraction and a motion generated by a non-local area contraction. In other embodiments, this determining step includes accounting for a global heart motion.

The method can also include the step of labeling the first portion of the heart whose motion is delayed with respect to other portions of the heart on the first set of images.

In some embodiments, the last three steps (i.e., using the ultrasound probe to obtain a second set of images of the patient's heart; determining, based on the second set of images, whether motion of the first portion of the heart is sufficiently synchronized with respect to other portions of the heart; and if it is determined in the determining step, that the motion of the first portion of the heart is not sufficiently synchronized, re-positioning the electrode at a second location and applying pulses to the electrode) are repeated until the first portion of the heart is sufficiently synchronized with respect to other portions of the heart.

In some embodiments, the first set of images and the second set of images are obtained at least 50 frames per second.

In some embodiments, the step of positioning a first electrode at a first location near the first portion of the patient's heart further includes positioning a second electrode at a third location of the patient's heart and applying pulses to the second electrode that are timed with respect to beating of the heart to attempt to advance, in time, the motion of the portion of the heart.

In some embodiments, the step of determining a first portion of the heart whose motion is delayed with respect to other portions of the heart includes capturing a set of ultrasound image frames of a patient's cardiac cycle. The method can also include identifying pixels in the captured set of frames that correspond to a structure. In some embodiments, the method includes generating a set of output frames, wherein each output frame within the set of output frames is generated by selecting a first frame of the captured set; selecting a second frame of the captured set, wherein the second frame is subsequent in time to the first frame; setting pixels of the output frame that correspond to the structure in the first frame and do not correspond to the structure in the second frame to a first color; setting pixels of the output frame that correspond to the structure in the second frame and do not correspond to the structure in the first frame to a second color; and setting pixels of the output frame that correspond to the structure in the first frame and also correspond to the structure in the second frame to a third color. In some embodiments, the method includes displaying the output frames.

In some embodiments, the step of determining a first portion of the heart whose motion is delayed with respect to other portions of the heart includes capturing a set of ultrasound image frames of a patient's cardiac cycle. The method can also include identifying pixels in the captured set of frames that correspond to a structure. In some embodiments, the method includes generating a set of output frames, wherein each output frame within the set of output frames is generated by selecting a first frame of the captured set; selecting a second frame of the captured set, wherein the second frame is subsequent in time to the first frame; coloring pixels of the first frame that correspond to the structure a first color; coloring pixels of the second frame that correspond to the structure a second color; and overlaying the colorized first frame and the colorized second frame to generate the output frame. The method can also include displaying the output frames.

Another aspect of the invention relates to a method for generating an enhanced ultrasound display. The method includes capturing a set of ultrasound image frames and identifying pixels in the captured set of frames that correspond to a structure. The method also includes generating a set of output frames, wherein each output frame within the set of output frames is generated by selecting a first frame of the captured set; selecting a second frame of the captured set, wherein the second frame is subsequent in time to the first frame; coloring pixels of the first frame that correspond to the structure a first color; coloring pixels of the second frame that correspond to the structure a second color; and overlaying the colorized first frame and the colorized second frame to generate the output frame. The method also includes displaying the output frames.

In some embodiments, output frame consists of the first color, the second color and a third color. The third color can be generated by the overlap of the first color and the second color. In some embodiments, the third color indicates that an ultrasound scatterer is present at the same pixel location in both the first frame and the second frame.

The set of ultrasound image frames can be images of a beating heart. The structure can be a wall of a left ventricle.

In some embodiments, the captured set of frames is captured at least 50 frames per second.

In other embodiments, the first color and the second color are not applied to pixels in low intensity regions.

Another aspect of the invention relates to an enhanced ultrasound display. The method includes capturing a set of ultrasound image frames. The method also includes identifying pixels in the captured set of frames that correspond to a structure. The method can also include generating a set of output frames, wherein each output frame within the set of output frames is generated by selecting a first frame of the captured set; selecting a second frame of the captured set, wherein the second frame is subsequent in time to the first frame; setting pixels of the output frame that correspond to the structure in the first frame and do not correspond to the structure in the second frame to a first color; setting pixels of the output frame that correspond to the structure in the second frame and do not correspond to the structure in the first frame to a second color; and setting pixels of the output frame that correspond to the structure in the first frame and also correspond to the structure in the second frame to a third color. The method also includes displaying the output frames.

In some embodiments, the set of ultrasound image frames are images of a beating heart. The structure can be a wall of a left ventricle.

In some embodiments, the captured set of frames are captured at least 50 frames per second.

In some embodiments, the first color and the second color are not applied to pixels in low intensity regions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart depicting a method of positioning an electrode for improved cardiac synchronization, according to an illustrative embodiment of the invention.

FIG. 2 is a flow chart depicting a method for generating an enhanced ultrasound display, according to an illustrative embodiment of the invention.

FIG. 3 is a schematic illustration of a heart indicating lead placement for cardiac resynchronization therapy, according to an illustrative embodiment of the invention.

FIG. 4 is a schematic illustration of how the overlapping colors are generated on a display, according to an illustrative embodiment of the invention.

FIG. 5 is an enhanced ultrasound image, according to an illustrative embodiment of the invention.

FIG. 6A is a processed ultrasound image frame and time t=0 ms, according to an illustrative embodiment of the invention.

FIG. 6B is a processed ultrasound image frame and time t=20 ms, according to an illustrative embodiment of the invention.

FIG. 6C is a processed ultrasound image frame and time t=40 ms, according to an illustrative embodiment of the invention.

FIG. 6D is a processed ultrasound image frame and time t=60 ms, according to an illustrative embodiment of the invention.

FIG. 6E is a processed ultrasound image frame and time t=80 ms, according to an illustrative embodiment of the invention.

FIG. 6F is a processed ultrasound image frame and time t=100 ms, according to an illustrative embodiment of the invention.

FIG. 7 is a processed ultrasound image including physician identified motion, according to an illustrative embodiment of the invention.

FIG. 8A is a processed ultrasound image with motion identified and labeled at time t=0 ms, according to an illustrative embodiment of the invention.

FIG. 8B is a processed ultrasound image with motion identified and labeled at time t=20 ms, according to an illustrative embodiment of the invention.

FIG. 8C is a processed ultrasound image with motion identified and labeled at time t=40 ms, according to an illustrative embodiment of the invention.

FIG. 8D is a processed ultrasound image with motion identified and labeled at time t=60 ms, according to an illustrative embodiment of the invention.

FIG. 8E is a processed ultrasound image with motion identified and labeled at time t=80 ms, according to an illustrative embodiment of the invention.

FIG. 8F is a processed ultrasound image with motion identified and labeled at time t=100 ms, according to an illustrative embodiment of the invention.

FIG. 9A is a processed ultrasound image with a mechanical activation map, according to an illustrative embodiment of the invention.

FIG. 9B is a mechanical activation map, according to an illustrative embodiment of the invention.

FIG. 9C is a mechanical activation map with time labeled, according to an illustrative embodiment of the invention.

FIG. 9D is a mechanical activation map, according to an illustrative embodiment of the invention.

FIG. 9E is a mechanical activation map, according to an illustrative embodiment of the invention.

### DETAILED DESCRIPTION OF THE PREFERRED EMBODIMENTS

The inventors have recognized that evaluating the performance of the heart in real time while pulses are being applied to the electrode using direct cardiac imaging should significantly improve the electrode-positioning procedure. US2005/0143657 (application Ser. No. 10/996,816, filed Nov. 24, 2004), which is incorporated herein by reference, discloses a miniature probe that can be used to obtain video images of the heart in real time without anesthesia or with minimal anesthesia. In particular, the '816 application discloses obtaining video images of the heart in real time using transesophageal echocardiography (“TEE”). A preferred view for implementing this TEE is the transgastric short axis view (“TGSAV”) of the LV of the heart.

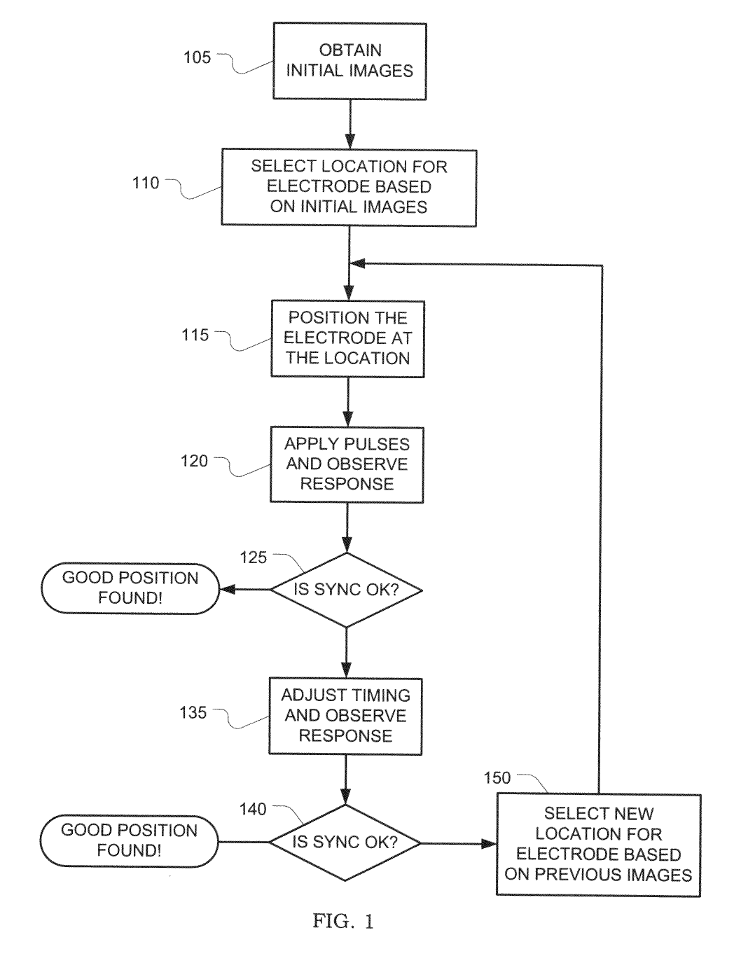

FIG. 1 is a flow chart depicting a method of positioning an electrode for improved cardiac synchronization, according to an illustrative embodiment of the invention. First, in step 105, a moving video image of operation of the heart is obtained. This moving video image includes a plurality of frames that are taken in rapid sequence (e.g., at 50 or at 60 frames per second). The moving video images can be obtained by inserting an ultrasound probe, for example, the ultrasound probe of US2005/0143657, into a patient's esophagus, and using that probe to obtain the images. The ultrasound probe can be used to obtain a first set of images of the patient's heart. These images are then displayed. The images that are displayed may be conventional moving video ultrasound images. Optionally, they may be enhanced using the techniques described below or using other techniques.

In step 110, the operator selects a location where an electrode should be placed based on the video images obtained in step 105. The electrode may be any conventional electrode that is used for traditional cardiac resync therapy. The location where the electrode is to be positioned may be selected based on identifying which portion of the heart contracts last, and selecting a position in the vicinity of that portion of the heart.

Next, in step 115, the electrode is placed at the selected location, or as close as possible to the selected location. To get the electrode to a desired position for endocardial lead positioning, the electrode may be inserted into the coronary sinus and then a branch of the coronary sinus to a first position in the heart using conventional approaches that are well known to persons skilled in the relevant arts. For subsequent positioning, discussed below, the electrode may be advanced, backed up, steered, etc. to get it to the new position. If the lead is being placed epicardially, again an initial position will be selected and then subsequent positions selected.

After the electrode is positioned, pulses are applied to the electrode in step 120. The pulses are timed with respect to beating of the heart to attempt to advance, in time, the motion of the portion of the heart. While the pulses are applied, new moving video images are obtained and displayed. Optionally, those images are enhanced to show movement as described below. Those images are observed to determine how the heart operates when the pulses are applied.

In step 125, a determination is then made as to whether the motion of the first portion of the heart is sufficiently synchronized with respect to the other portions of the heart. If adequate synchronization is obtained, a good position has been found, and the process stops and the electrode is left in place.

If the result of the determination of step 125 is that adequate synchronization has not been achieved, then the process continues in step 135, where the timing of the pulses applied to the electrode is adjusted to try to improve synchronization. While the pulses are being adjusted, new moving images are obtained and the operation of the heart is observed on the display. Based on these displayed images, the operator can determine, in step 140, if adequate synchronization has been achieved. If adequate synchronization is obtained, a good position has been found. The process then stops and the electrode can be left in place.

If it is determined, in step 140, that adequate synchronization has not been achieved, a new position for the electrode is selected in step 150. The electrode can be re-positioned at a new location, and the steps subsequent to step 115 may be implemented as many times as desired to try to achieve adequate synchronization.

Determining whether the heart is sufficiently synchronized is a judgment determination that the presiding physician will have to make. While minimal dyssynchrony is a desirable objective, a certain level of dyssynchrony may be acceptable. For example, in some situations a heart may be deemed sufficiently synchronized when the total dyssynchrony delay is about 20 ms to about 40 ms. In other situations, the physician may determine that a 60 ms delay is the best that can be done for a particular patient. Those of skill in the art will realize that whether a heart is sufficiently synchronized may depend on the particular circumstances of the patient.

Optionally (as in conventional CRT), more than one electrode may be used to improve cardiac synchronization. For example, a second electrode can be positioned at a location of the patient's heart that is spaced apart from the first electrode. Pulses can be applied to the second electrode that are timed with respect to beating of the heart to attempt to advance, in time, the motion of the delayed portion of the heart. The first and second electrodes can be pulsed at the same time or the first and second electrodes can be pulsed at varying times to try to reduce the dyssynchrony.

Note that each of the selected locations to place the electrode may be chosen by the operator based on all the previously obtained images of the patient. Preferably, imaging is implemented in real time. The use of real-time imaging will allow visual assessment of wall motion, assessment of key parameters of cardiac performance such as left ventricular end-diastolic area, left ventricular end-systolic area, and fractional area change, and, most importantly, the effects of stimuli from the currently selected lead placement. In particular, real-time imaging at suitably high frame rates, for example, 50 frames per second or faster, will allow easy visual determination of the timing of the development mechanical activation up to the corresponding precision limits. Note that at 50 frames per second, a new frame is obtained every 20 milliseconds, which provides an adequate resolution in time to monitor cardiac performance.

Optionally, suitable spatio-temporal image processing such as automated detection of the difference between two successive images, may be used to enhance the ability of the operator to visually determine timing of the development mechanical activation, and the presence or absence of significant dyssynchrony. Suitable approaches for implementing such processing are described below. Optionally, motion detection may distinguish active versus passive motion, i.e., motion generated by contraction of the local area equal to a region or segment (“local contraction”) versus motion generated by contraction of other areas, for example, rotation, or non-local area contraction. Optionally, local area motion may be tracked, preferably in a Lagrangian coordinate system as opposed to an Eulerian coordinate system. Optionally, artifacts induced by global heart motion (for example, rotation, longitudinal motion) are accounted for. An index such as LV cavity height+LV cavity width may be useful for this purpose. Optionally, correlation from speckle tracking, especially detection of simultaneous circumferential contraction and radial thickening in the same local area may be implemented. Optionally, qualitative visual information may be provided. For example, simultaneous, synchronized playback of two video loops may be implemented, optionally with overlays of LV border at end diastole, LV border at end systole, or semi-transparent overlay of border sequence from one loop on top of a second loop. One of the loops can be displayed in real time. Optionally, the images may be used to determine how well single lead pacing is working, in order to determine whether a bi-ventricular device should be installed.

Suitable approaches to enhance the ability of the operator to visually determine timing of the development mechanical activation, and the presence or absence of significant dyssynchrony, are described below. This approach, which is referred to herein as “mechanical activation mapping” is particularly useful because the mechanical activation information can be overlaid on the same display on top of other information about cardiac function, including (a) other wall motion abnormalities; (b) presence of scar tissue; (c) other wall defects such as thickening; and (d) measures of cardiac function such as left ventricular end-diastolic area, left ventricular end-systolic area, and fractional area change. One preferred way to overlay the mechanical activation information on top of the other information is using color, as described below. Displaying this additional information simultaneously allowed the physician to optimize lead placement for CRT even in the presence of other cardiac defects.

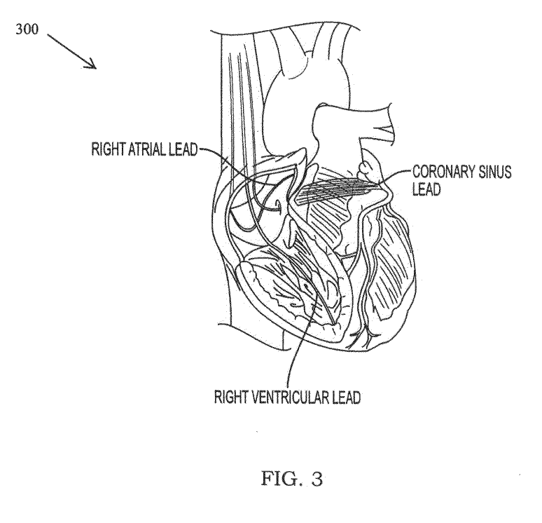

FIG. 3 is a schematic illustration of a heart 300 indicating lead placement for cardiac resynchronization therapy, according to an illustrative embodiment of the invention. FIG. 3 shows lead placement, for example, right atrial lead, coronary sinus lead, and right ventricular lead, for a biventricular pacemaker. The overall goal of cardiac resynchronization therapy in a healthy heart may be to effectively mimic “normal” stimulation of the left ventricle from the His-Purkinje network (FIG. 1) by appropriately timed stimuli at two sites (or potentially more than two sites) generated by a cardiac pacemaker (biventricular pacemakers typically stimulate at two sites—FIG. 3). Endpoint 3 addresses the overall effectiveness of the stimulation location and timing, and endpoint 2 addresses how we get there by identifying asynchrony arising from a given pattern of stimulation sites and timing.

The approach described herein focuses on endpoint 2: appropriate, coordinated mechanical activation. This can be a better endpoint than endpoint 1 for the purposes of assessing cardiac dynamics, since it is closer to the goal of efficient ejection (endpoint 3). Endpoint 3 is also addressed in application Ser. No. 10/996,816, filed Nov. 24, 2004, which is incorporated herein by reference, and discloses a miniature probe that can be used to obtain video images of the heart, in real time without anesthesia or with minimal anesthesia. In particular, the '816 application discloses obtaining video images of the heart in real time using transesophageal echocardiography (“TEE”). A preferred view for implementing this TEE is the TGSAV of the LV of the heart.

Endpoints 1 and 2 can be measured by activation mapping, that is, the display of the progress of waves of activation (electrical activation in the case of endpoint 1, mechanical activation in the case of endpoint 2) across the left ventricle. In this context, endpoint 2 is especially important, since mechanical activation mapping, preferably in real time, can identify areas to be addressed (inappropriate or delayed wall motion) in order to improve ejection.

Ultrasound imaging (e.g., using a miniaturized TEE probe such as the one described in the '816 application) may offer several significant advantages. There is adequate time resolution when image frames are acquired at 50 or 60 frames per second (fps) for bursts of 3 seconds, offering a 20 ms time resolution for typically 3 or more cardiac cycles. For systems with time constants in tissue>>3 seconds, the limiting factor is the number of frames in a burst, thus, for example, 16.7 ms time resolution would be achieved at 60 fps, and a 2.5 second burst would typically offer more than two cardiac cycles. Faster rates could be achieved with a cool-down period before a burst by operating at a lower frame rate.

There is also adequate spatial resolution. Consider the following sample calculation at 6 cm depth. The probe described in the '816 application yields resolution cells in 2D of 300 μM (axial)×4 mm (azimuthal), and resolution cells in 3D of 300 μM (axial)×4 mm (azimuthal)×2 mm (elevation), for a volume of 2.4 mm³. This is 1/100 of the size of the volume for motion detection by tagged MRI in Wyman et al., namely 0.25 cm³. The use of this resolution for motion detection by ultrasound would thus offer equivalent precision in activation mapping to that offered by tagged MRI, already shown to be adequate. Moreover, motion in systole may represent typically 1 cm over 200 ms. Then at 16.7 ms time resolution, one expects motion of 833 μm, greater than the axial resolution of 300 μm. Thus, one should be able to easily detect axial motion, which corresponds to circumferential motion for the critical septal and free walls.

Finally, using TEE makes it easy to obtain real-time information about ejection (such as end systolic area, estimated end systolic volume, fractional area change, estimated ejection fraction) during pacemaker implantation, because ejection fraction can be computed readily from 2D ultrasound images of the TGSAV of the LV. One suitable approach for computing the Ejection Fraction is described below with reference to EQNS. 1-2.

Quantification or semiquantification of left ventricular ejection fraction is routinely performed by several two-dimensional echocardiographic techniques. Mid-left ventricular end-diastolic and end-systolic diameters can be measured by using the M-mode cursor, oriented by two-dimensional imaging, to ensure appropriate positioning of the line of measurement, generally at the mid-papillary muscle level from the short (transverse cardiac) axis image. The left ventricular end-diastolic diameter (LVEDD) is measured as coincident to the R wave of the electrocardiogram, and the left ventricular end-systolic diameter (LVESD) is measured at the maximal excursion of the septum during the cardiac cycle. The ejection fraction (EF) is calculated by using the square of these diameters (EQN. 1):

EF (%)=[(LVEDD)²−(LVESD)²]×100/(LVEDD)². EQN. 1

A similar evaluation can be made by estimating the end-diastolic and end-systolic volumes provided by these diameters [Teichholz L E, Kreulen T, Herman M V, Gorlin R. Problems in Echocardiographic Volume Determinations: Echocardiographic-Angiographic Correlations in the Presence of Asynergy. Am J Cardiol 1976; 37:7-11]. The method based on the squared diameters is clinically satisfactory but can be limited by the presence of regional wall motion abnormalities, especially at levels near the base and the apex of the left ventricle, and it implies certain a priori assumptions about overall left ventricular shape. Additionally, it does not incorporate changes in the long-axis length of the left ventricle during contraction, which can contribute to errors from this calculation, although a correction can be modeled into the original equation [Quinones M A, Waggoner A D, Reduto L A, Nelson J G, Young J B, Winters W L Jr., et al. A New, Simplified and Accurate Method for Determining Ejection Fraction with Two-Dimensional Echocardiography. Circulation 1981; 64:744-753]”; Rumebrger J A et al., Determination of Ventricular Ejection Fraction: A Comparison of Available Imaging Methods, Mayo Clin Proc. 1997; 72:360-370.

For example, the Teichholz method estimates the left-ventricular volume V (in cm³) from the diameter (in cm) from EQN. 2.

V=d³×(7/(2.4+d)). EQN. 2

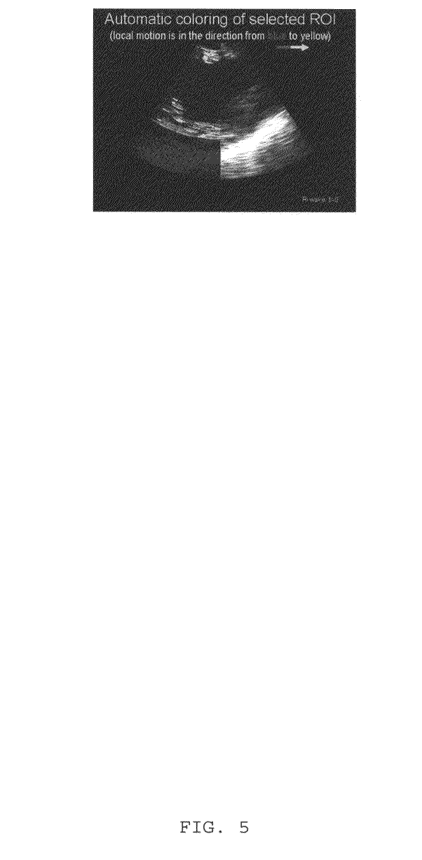

Applying the Teichholz method to FIG. 5 above yields a left ventricular end-diastolic volume of 44 cm³, a left ventricular end-systolic volume of 18 cm³, and an ejection fraction of 59% from end-diastolic and end-systolic diameters of 3.3 cm and 2.3 cm, respectively.

Optionally, real-time information about ejection (such as end systolic area, estimated end systolic volume, fractional area change, and estimated ejection fraction) may be displayed during pacemaker implantation.

One suitable approach can comprise the following steps: (1) marking a fiducial point (R-wave, pacing signal, etc.) accurately on a sequence of ultrasound images (frames), thus defining the start of a cardiac cycle; (2) acquiring a sequence of frames including a cardiac cycle and a sufficient number of frames before the start of that cardiac cycle for the steps below; (3) for each frame, suitably coloring that frame and one or more preceding frames, and then compounding the colored frame and preceding frames so as to obtain a sequence of compounded colored frames covering a cardiac cycle with the fiducial point marked, each compounded colored frame indicative of cardiac wall motion, as described herein; and (4) providing a means for an operator to play the sequence of compounded colored frames indicative of cardiac wall motion and mark the frames corresponding to the onset of cardiac motion, in particular by sectors, so as to obtain an activation sequence indicative of the onset of mechanical activation in each of the sectors where the operator indicates the onset of motion.

Often, part or all of the wall motion between two consecutive ultrasound images of a cardiac cycle can be difficult to detect and visualize, due to the little movement and/or faintness, especially for the side wall. The following is a suitable approach for emphasizing such motion on the images, so that to facilitate the study and diagnosis. This approach relies on the concept of additive color mixing of the RGB color model. For example, mixing red and green will generate yellow, or mixing blue and yellow will produce white.

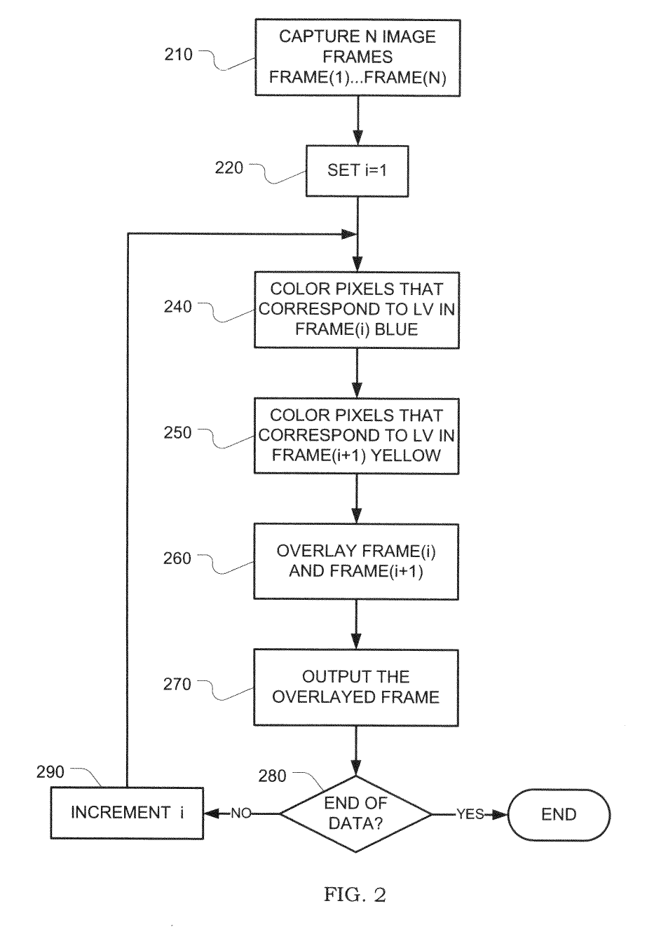

FIG. 2 is a flow chart depicting a suitable method for generating an enhanced ultrasound display that highlights motion of the relevant structures. Although it is described in the context of images of a beating heart and a cardiac cycle, it can be used in other contexts as well besides cardiac applications. First, in step 210, N image frames are captured. Those image frames are referred to herein as frame(1) . . . frame(N). Preferably, enough image frames are captured to include at least one complete cardiac cycle, at a frame rate that is sufficiently high to resolve the relevant data (e.g., three seconds of data at 50 frames per second or more).

In step 220, a loop is initialized by setting a pointer i to 1. Then, in step 240, the pixels of frame(i) that correspond to the relevant structure are set to a first color. For example, the pixels that correspond to the LV may be set to blue. In step 250, the pixels of frame(i+1), which is the next frame in time that follows frame(i), that correspond to the same structure are set to a second color. In our example, the pixels that correspond to the LV are set to yellow in step 250. Conventional algorithms for distinguishing what portions of the image correspond to the relevant structure and what portions of the image correspond to speckle or noise may be used. One way to implement this is not to apply the color to pixels in low intensity regions.

In step 260, frame(i) and frame(i+1) are overlayed to generate an output frame. The result of compounding these two frames is an output frame with three colors, with the third color resulting from mixing of two colors used to colorize frame(i) and frame(i+1). For example, if structures in frame(i) are colored blue and structures in frame(i+1) are colored yellow, then the processed compounded frame (processed frame n) will have 3 colors: blue, yellow, and white, of varying intensities. The white regions indicate where the wall (and other scatterers) overlaps on the two unprocessed frames. The blue and yellow regions indicate where the wall (and other scatterers) appear in only one of the two unprocessed frames. More specifically, blue regions indicate that the wall was present only in unprocessed frame(i), and yellow regions indicate that the wall was present only in frame(i+1). The resulting output frame can be used to indicate local wall motion, in the direction moving from the blue region to the yellow region. Preferably, low-intensity regions (typically in the cavity) are colored white instead of blue or yellow, because the apparent motion of speckle within the cavity is distracting. This may be done by not colorizing those pixels blue or yellow in the input frames (i.e., in steps 240 and 250), or by removing the colors after the output frame is generated in step 260.

In step 270, the output frame is displayed using any conventional display approach such as a conventional ultrasound display screen, or other type of display.

In step 280, a test is performed to see if the end of the data has been reached. If the end of data has been reached, the process ends. If additional data remains, the pointer i is incremented, and the process returns to step 240 to generate another output frame.

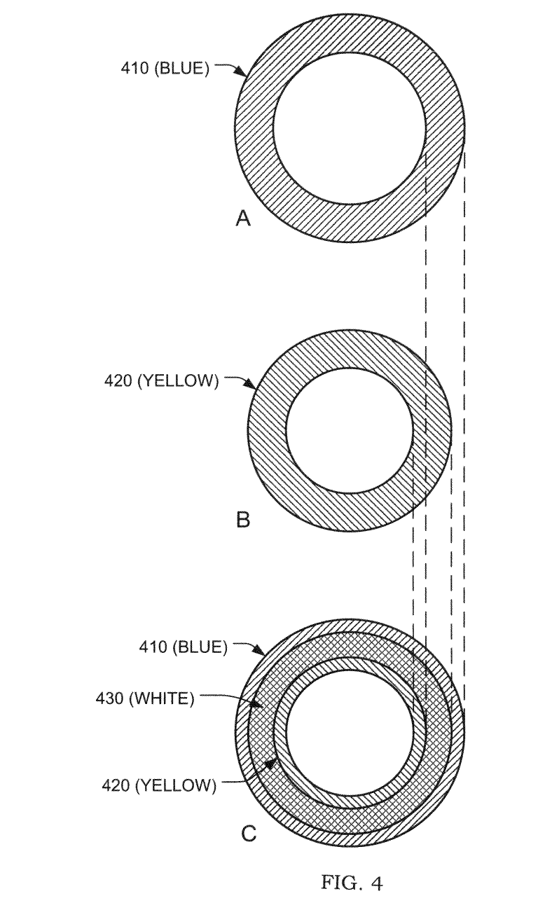

FIG. 4 is a schematic illustration of how the overlapping colors are generated on a display when the method of FIG. 2 is implemented. Panels A and B of FIG. 4 are schematic representations of images of the LV wall of a beating heart at two different times. For example at time t1 the position of the wall in the image may be as shown by region 410 in panel A. A short time later at t2, after the LV has contracted a small amount, the LV wall would move to a new state in the image as seen in panel B. (Note that the circles are slightly smaller, to indicate a contraction.) Note that these two panels (A and B) are schematic representations of two consecutive frames, frame(i) and frame(i+1) in the discussion of FIG. 2 above.

After the images are captured, the pixels in a first image shown in panel A that correspond to the LV wall (or other structure of interest) are colored a first color, for example blue (region 410). The pixels in a second image shown in panel B that correspond to the LV wall (or other structure of interest) are colored a second color, for example yellow (region 420). Note that this corresponds to steps 240 and 250 in the discussion of FIG. 2 above.

When the two images are overlaid or compounded, (a) pixels that corresponds to structure in the first image but do not correspond to the structure in the second image will show up as blue in the compounded image; (b) pixels that corresponds to structure in the second image but do not correspond to the structure in the first image will show up as yellow in the compounded image; and (c) pixels that corresponds to structure in both the first image and the second image will show up as white in the compounded image, because blue plus yellow forms white on a computer display. Note that this corresponds to step 260 in the discussion of FIG. 2 above. The compounded image therefore shows the motion of the structure, in the direction from blue to yellow (in this situation, the contraction of a heart wall in the direction of blue to yellow).

In all the embodiments described herein, the captured set of frames is preferably captured at least 50 frames per second (e.g., at 50 or 60 frames per second).

In practice, it is preferable for the still regions (i.e. overlapping regions) to be displayed in white colors as in original grey-scale image format. In addition, the regions with motion should be colored in a fashion that luminosities stay constant, in order to better distinguish the disappearing region and appearing region, which gives information about the wall motion. This processing may be implemented as part of steps 240 and 250 discussed above in connection with FIG. 2, and a preferred method for implementing this is described in more detail below, and includes the following steps.

For each pixel, if the pixel is faint in both image frames, the original intensity value on unprocessed frame n is used on the processed image, as in EQN. 3.

If Max(Iₙ₋₁( x̅),Iₙ( x̅))<thrs_int,

I′ₙ( x̅,R)=Iₙ(x)

then I′ₙ(x,G)=I′ₙ(x,R)

I′ₙ(x,B)=I′ₙ( x̅,R) EQN. 3

If the intensity of the pixel only changes a little between the 2 frames, the original intensity value on unprocessed frame n is used on the processed image, as in EQN. 4.

If |Iₙ₋₁( x̅)−Iₙ( x̅)|<thrs_diff,

I′ₙ( x̅,R)=Iₙ( x̅)

then I′ₙ( x̅,G)=I′ₙ( x̅,R)

I′ₙ( x̅,B)=I′ₙ( x̅,R) EQN. 4

If the pixel intensity is above a certain threshold in at least one frame and the intensity difference between the two frames is above a certain threshold, we consider this pixel as a pixel belonging to a region undergoing motion. Then, if the pixel intensity is larger in unprocessed frame N (compared to that of the previous unprocessed frame) we color the pixel yellow (by setting R and G to the maximum); and if the pixel intensity is smaller in unprocessed frame N (compared to that of the previous unprocessed frame) we color the pixel blue (by setting B to the maximum and G to 50%). If neither of those conditions is satisfied, the pixels are not colored, as in EQNS. 5-6.

$$\begin{matrix}If \{ \begin{matrix}Max ( {I}_{n - 1} ( \overline{x} ) , {I}_{n} ( \overline{x} ) ) > thrs\_int \\ \vert {I}_{n - 1} ( \overline{x} ) - {I}_{n} ( \overline{x} ) \vert > thrs\_diff \\ {I}_{n - 1} ( \overline{x} ) - {I}_{n} ( \overline{x} ) < 0\end{matrix} , \\ then \begin{matrix}{I}_{n}^{′} ( \overline{x} , R ) = 255 \\ {I}_{n}^{′} ( \overline{x} , G ) = 255 \\ {I}_{n}^{′} ( \overline{x} , B ) = 0\end{matrix} & EQN . 5 \\ If \{ \begin{matrix}Max ( {I}_{n - 1} ( \overline{x} ) , {I}_{n} ( \overline{x} ) ) > thrs\_int \\ \vert {I}_{n - 1} ( \overline{x} ) - {I}_{n} ( \overline{x} ) \vert > thrs\_diff \\ {I}_{n - 1} ( \overline{x} ) - {I}_{n} ( \overline{x} ) > 0\end{matrix} , \\ then \begin{matrix}{I}_{n}^{′} ( \overline{x} , R ) = 0 \\ {I}_{n}^{′} ( \overline{x} , G ) = 128 \\ {I}_{n}^{′} ( \overline{x} , B ) = 255\end{matrix} & EQN . 6\end{matrix}$$

In EQNS. 3-6 above, x is the image pixel indexed by (x, y). Iₙ( x̅) denotes the intensity of this pixel in unprocessed frame n n, and Iₙ₋₁( x̅) is such value in the previous frame. I′ₙ( x̅, R) denotes the intensity value of red channel of pixel x̅ in the processed image. I′ₙ ( x̅, G) and I′ₙ( x̅, B) denote such value in green and black channel, respectively.

FIG. 5 is an enhanced ultrasound image, according to an illustrative embodiment of the invention. When the coloring scheme above is implemented, as illustrated in FIG. 5, the wall direction moves locally in a direction from blue to yellow.

Optionally, feature tracking of wall regions and/or morphological analysis to enhance image quality and visual appearance may be implemented

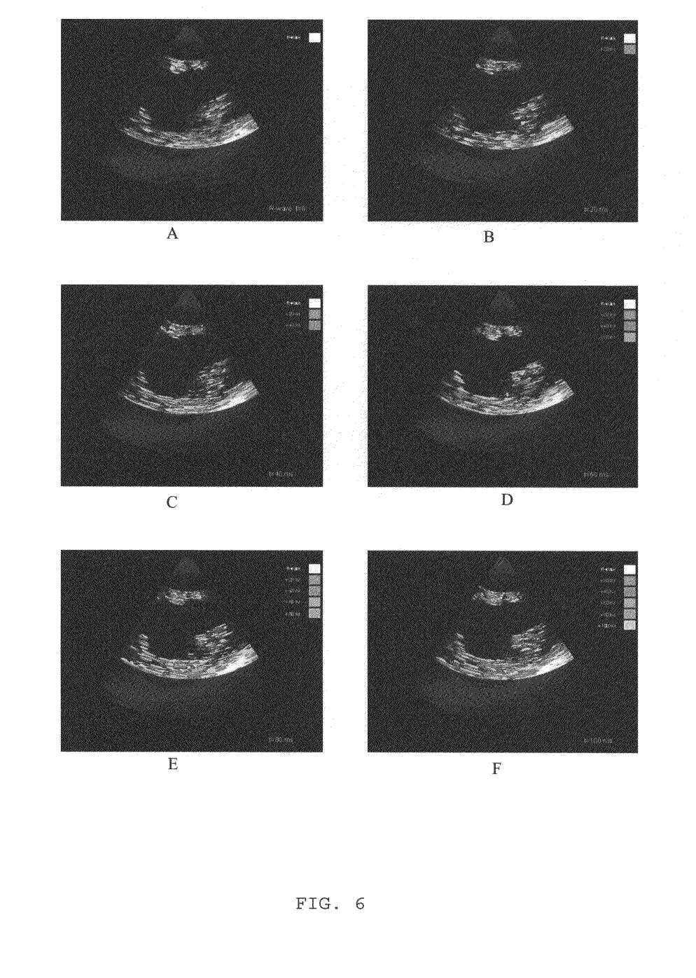

FIGS. 6A-F show six processed sample frames, at 20 ms intervals, with the first frame occurring at the time of the R-wave, automatically colored using the same coloring scheme discussed above with reference to FIG. 5. The motion is shown in the direction of blue to yellow.

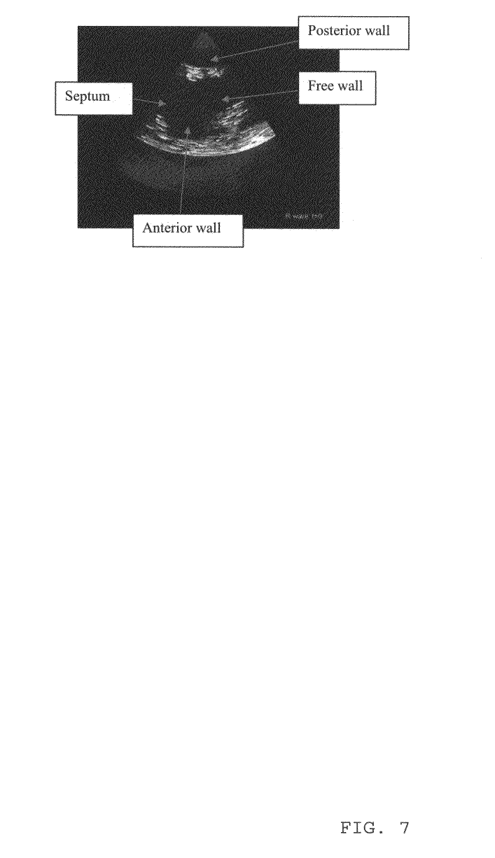

FIG. 7 is a processed ultrasound image including physician identified motion, according to an illustrative embodiment of the invention. The physician identifies motion by comparing successive frames, and optionally labels what he/she sees. For example, the physician can label the septum, the posterior wall, the free wall and/or the anterior wall.

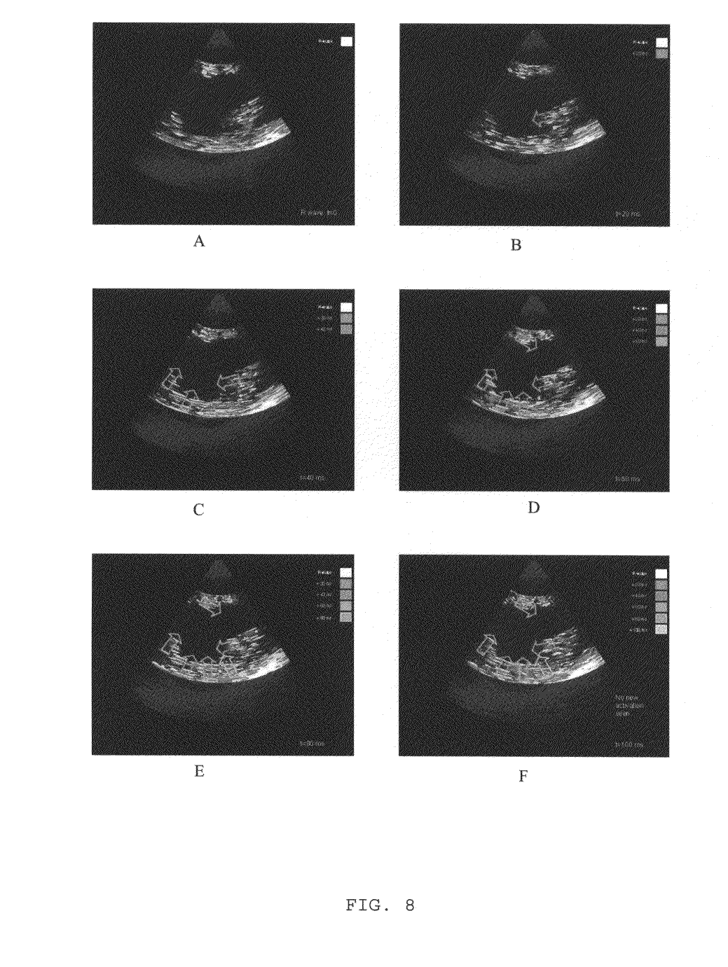

FIGS. 8A-F show a sequence of six colored frames at 20 ms intervals, with the first frame occurring at the time of the R-wave, with motion identified and labeled by a physician. The identified motion is indicated on the display using a suitable user interface. In this example, we see that the motion begins in the anterior part of the free wall at time 20 ms (FIG. 8B), is next seen in the anterior wall and the very anterior part of the septum at time 40 ms (FIG. 8C), then progresses to the middle of the anterior wall at time 60 ms (FIG. 8D), and finally progresses to the free wall side of the anterior wall at time 80 ms (FIG. 8E).

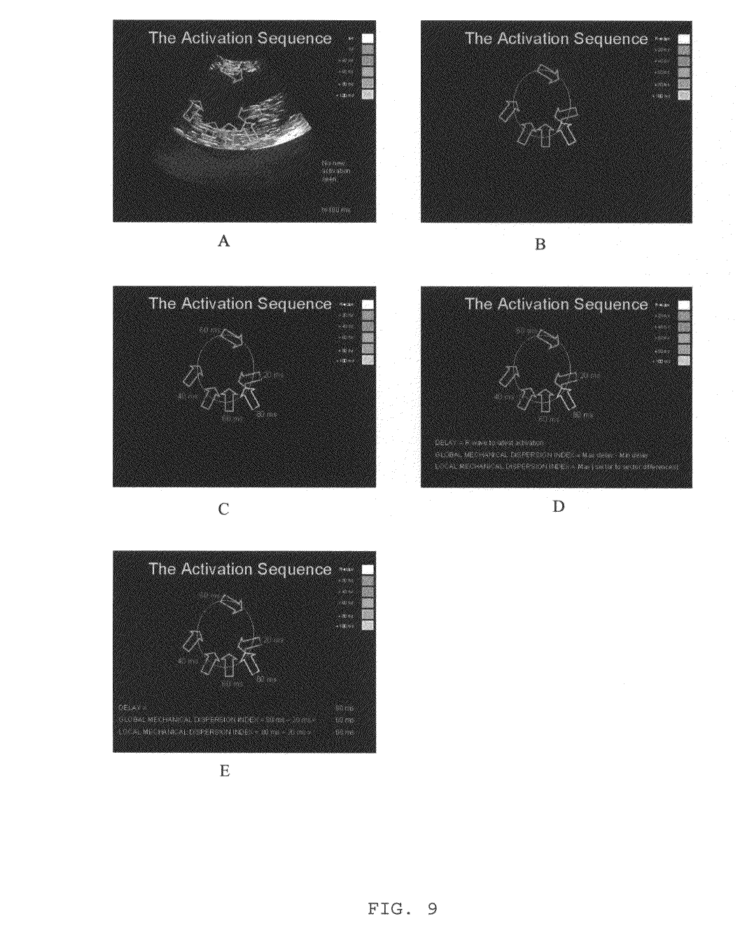

One can visualize the required mechanical activation map by playing the above sequence of frames, for example, as a slow-motion video. FIGS. 9A-E are processed ultrasound images with a mechanical activation map, according to an illustrative embodiment of the invention. The mechanical activation map may also be generated automatically from the sequence of labelings, for example arrows, as shown in FIGS. 9A-E.

Three useful and illustrative indices have been identified EQNS. 7.

DELAY=R wave to latest activation

GLOBAL MECHANICAL DISPERSION INDEX=Max delay−Min delay

LOCAL MECHANICAL DISPERSION INDEX=Max{sector to sector differences} EQN. 7

Other indices are readily constructed from the activation sequence shown in FIG. 9C and EQN. 8 below. The values of these indices in this example are:

DELAY=80 ms

GLOBAL MECHANICAL DISPERSION INDEX=80 ms−20 ms=60 ms

LOCAL MECHANICAL DISPERSION INDEX=80 ms−20 ms=60 ms EQN. 8

Note in particular the LOCAL MECHANICAL DISPERSION INDEX=60 ms quantifies the sharp asynchrony in mechanical activation as one moves from the anterior wall to the free wall.

After the delays and asynchrony are observed using this approach, the physician will be better able to select an appropriate portion for the electrode, as described above in connection with FIG. 1.

An alternative embodiment is similar to FIG. 2, except steps 240-270 are replaced by the following three steps: (1) pixels of the output frame that correspond to the structure in frame(i) and do not correspond to the structure in frame(i+1) are set to a first color; (2) pixels of the output frame that correspond to the structure in frame(i+1) and do not correspond to the structure in the frame(i) are set to a second color; and (3) pixels of the output frame that correspond to the structure in frame(i) and also correspond to the structure in frame(i+1) are set to a third color. This achieves a similar end result to the steps 240-270, but does not rely on overlaying. The first, second, and third colors should all be different, and are preferably easily distinguished. The third color is preferably white or grey because it is not used to indicate motion. The output frames are eventually displayed in any conventional manner, for example on an ultrasound machine or other suitable display screen.

Note that all the methods described above are preferably implemented using conventional microprocessor-based hardware, e.g., on a computer or a dedicated ultrasound machine that has been programmed to carry out the steps of the various methods, and display the output frames (using, e.g., conventional display hardware).

Variations, modifications, and other implementations of what is described herein will occur to those of ordinary skill in the art without departing from the spirit and the scope of the invention as claimed. Accordingly, the invention is to be defined not the preceding illustrative description but instead by the spirit and scope of the following claims.

What is claimed is:

1. A method for positioning an electrode for improved cardiac synchronization comprising the steps of:
(a) inserting an ultrasound probe into a patient's esophagus;
(b) using the ultrasound probe to obtain a first set of images of the patient's heart;
(c) determining, based on the first set of images, a first portion of the heart whose motion is delayed with respect to other portions of the heart;
(d) positioning a first electrode at a first location near the first portion of the patient's heart;
(e) applying pulses to the first electrode that are timed with respect to beating of the heart to attempt to advance, in time, the motion of the portion of the heart;
(f) using the ultrasound probe to obtain a second set of images the patient's heart;
(g) determining, based on the second set of images, whether motion of the first portion of the heart is sufficiently synchronized with respect to other portions of the heart; and
(h) if it is determined, in step (g), that the motion of the first portion of the heart is not sufficiently synchronized with respect to other portions of the heart, re-positioning the first electrode at a second location and applying pulses to the first electrode that are timed with respect to beating of the heart to attempt to advance, in time, the motion of the portion of the heart.

2. The method of claim 1 further comprising the step of processing the first set of images and the second set of images to highlight portions of the heart that have moved between two successive images in the first set of images.

3. The method of claim 2 wherein the first set of images and the second set of images are enhanced with at least two colors.

4. The method of claim 1 wherein the processing includes detecting a difference between two successive images in the first set of images.

5. The method of claim 1 wherein step (c) comprises the step of distinguishing between a motion generated by a local area contraction and a motion generated by a non-local area contraction.

6. The method of claim 1 wherein step (c) comprises accounting for a global heart motion.

7. The method of claim 1 further comprising the step of labeling the first portion of the heart whose motion is delayed with respect to other portions of the heart on the first set of images.

8. The method of claim 1 wherein steps (f)-(h) are repeated until the first portion of the heart is sufficiently synchronized with respect to other portions of the heart.

9. The method of claim 1 wherein the first set of images and the second set of images are obtained at least 50 frames per second.

10. The method of claim 1 wherein step (d) further comprises positioning a second electrode at a third location of the patient's heart and applying pulses to the second electrode that are timed with respect to beating of the heart to attempt to advance, in time, the motion of the portion of the heart.

11. The method of claim 1, wherein the step of determining a first portion of the heart whose motion is delayed with respect to other portions of the heart comprises the steps of:
capturing a set of ultrasound image frames of a patient's cardiac cycle;
identifying pixels in the captured set of frames that correspond to a structure;
generating a set of output frames, wherein each output frame within the set of output frames is generated by
selecting a first frame of the captured set,
selecting a second frame of the captured set, wherein the second frame is subsequent in time to the first frame,
setting pixels of the output frame that correspond to the structure in the first frame and do not correspond to the structure in the second frame to a first color,
setting pixels of the output frame that correspond to the structure in the second frame and do not correspond to the structure in the first frame to a second color, and
setting pixels of the output frame that correspond to the structure in the first frame and also correspond to the structure in the second frame to a third color; and
displaying the output frames.

12. The method of claim 1, wherein the step of determining a first portion of the heart whose motion is delayed with respect to other portions of the heart comprises the steps of:
capturing a set of ultrasound image frames of a patient's cardiac cycle;
identifying pixels in the captured set of frames that correspond to a structure;
generating a set of output frames, wherein each output frame within the set of output frames is generated by
(i) selecting a first frame of the captured set,
(ii) selecting a second frame of the captured set, wherein the second frame is subsequent in time to the first frame,
(iii) coloring pixels of the first frame that correspond to the structure a first color,
(iv) coloring pixels of the second frame that correspond to the structure a second color, and
(v) overlaying the colorized first frame and the colorized second frame to generate the output frame; and
displaying the output frames.

13. A method for generating an enhanced ultrasound display, the method comprising the steps of:
capturing a set of ultrasound image frames;
identifying pixels in the captured set of frames that correspond to a structure;
generating a set of output frames, wherein each output frame within the set of output frames is generated by
(a) selecting a first frame of the captured set,
(b) selecting a second frame of the captured set, wherein the second frame is subsequent in time to the first frame,
(c) coloring pixels of the first frame that correspond to the structure a first color,
(d) coloring pixels of the second frame that correspond to the structure a second color, and
(e) overlaying the colorized first frame and the colorized second frame to generate the output frame; and
displaying the output frames.

14. The method of claim 13 wherein the output frame consists of the first color, the second color and a third color, the third color generated by the overlap of the first color and the second color.

15. The method of claim 14 wherein the third color indicates that an ultrasound scatterer is present at the same pixel location in both the first frame and the second frame.

16. The method of claim 13 wherein the set of ultrasound image frames are images of a beating heart.

17. The method of claim 16 wherein the structure is a wall of a left ventricle.

18. The method of claim 16 wherein the captured set of frames are captured at least 50 frames per second.

19. The method of claim 16, wherein the first color and the second color are not applied to pixels in low intensity regions.

20. A method for generating an enhanced ultrasound display, the method comprising the steps of:
capturing a set of ultrasound image frames;
identifying pixels in the captured set of frames that correspond to a structure;
generating a set of output frames, wherein each output frame within the set of output frames is generated by
(a) selecting a first frame of the captured set,
(b) selecting a second frame of the captured set, wherein the second frame is subsequent in time to the first frame,
(c) setting pixels of the output frame that correspond to the structure in the first frame and do not correspond to the structure in the second frame to a first color,
(d) setting pixels of the output frame that correspond to the structure in the second frame and do not correspond to the structure in the first frame to a second color, and
(e) setting pixels of the output frame that correspond to the structure in the first frame and also correspond to the structure in the second frame to a third color; and
displaying the output frames.

21. The method of claim 20 wherein the set of ultrasound image frames are images of a beating heart.

22. The method of claim 21 wherein the structure is a wall of a left ventricle.

23. The method of claim 21 wherein the captured set of frames are captured at least 50 frames per second.

24. The method of claim 20, wherein the first color and the second color are not applied to pixels in low intensity regions.
